(19) United States
(12) Patent Application Publication
(10) Pub. No.: US 2026/0220770 A1
(43) Pub. Date: Jul. 30, 2026

(54) TITER PROCESSING METHOD AND SYSTEM BASED ON DEEP LEARNING

(71) Applicant: Hospital for Skin Diseases, Institute of Dermatology, Chinese Academy of Medical Sciences & Peking, Nanjing (CN)

(72) Inventors: Tong LIN, Nanjing (CN); Yan HAN, Nanjing (CN); Yueping YIN, Nanjing (CN); Yin YANG, Nanjing (CN); Jiaxuan JIN, Nanjing (CN)

(21) Appl. No.: 19/117,356

(22) PCT Filed: Aug. 7, 2024
(86) PCT No.: PCT/CN2024/110373

§ 371 Date: Mar. 31, 2025

(30) Foreign Application Priority Data
Oct. 20, 2023 (CN) 202311372626.0

(51) Int. Cl. G06T 7/00; G06T 7/62; G06V 10/26; G06V 10/34; G06V 10/764; G06V 10/82; G16H 10/40; G16H 50/20

(52) U.S. Cl. CPC G06T 7/0012; G06T 7/62; G06V 10/26; G06V 10/34; G06V 10/764; G06V 10/82; G16H 10/40; G16H 50/20

(57) ABSTRACT

The present application discloses a titer processing method and system based on deep learning, which belong to the field of machine learning, and comprises: obtaining a first image of non-syphilis treponemal serological test results; judging the clarity of the test image and outputting a second image; applying a convolutional neural network on the second image for detecting circular targets and outputting the center coordinates and radius of the circular targets; segmenting the second image according to the center coordinates and radius of the detected circular targets to extract circular target regions; sorting the extracted circular target regions according to preset sorting rules; using a convolutional neural network to classify the sorted circular target regions and outputting a negative or positive result; calculating a corresponding titer of the negative or positive result.

### TECHNICAL FIELD

The present application relates to the field of machine learning, and more specifically to a titer processing method and system based on deep learning, which are used for the automatic processing of non-treponemal serological test results.

### BACKGROUND

In clinical medical testing, syphilis serological examination is an important method for assisting in the diagnosis of syphilis, including treponemal serological tests and non-treponemal serological tests. In the qualitative and quantitative detection of traditional non-treponemal serological tests, the interpretation of qualitative results for positivity and negativity, as well as the interpretation method for quantitative titers, usually rely on the observation and judgment of images by an examiner, which leads to issues of subjective error and low efficiency.

Non-treponemal serological testing is an important immunological test for aiding the initial diagnosis of disease activity and assess treatment efficacy. This test determines positivity by reading the carbon antigen particles formed after the reaction between serum and different antigens. However, manual visual interpretation of the cards has low efficiency, and there are variations in results among different operators.

A Chinese patent application with application number CN201910078013.3, published on Jun. 7, 2019, discloses an ANA fluorescence slide automatic recognition method based on machine learning and deep learning, which belongs to the technical field of machine learning and deep learning, and addresses the problems in the prior art where manual judgment of ANA fluorescence slides requires high personnel requirements and is prone to misjudgment. The present application is based on a machine learning model, which calculates a titer model through a dataset; calculates a karyotype model through a dataset based on a deep learning model; reads the ANA fluorescence slide to be recognized, namely the image to be recognized, and extracts features from the image to be recognized, obtaining multiple extracted feature values; inputs the multiple features into the titer model to obtain a predicted titer; inputs the image to be recognized into the karyotype model to obtain a predicted karyotype; and obtains a recognition result based on the predicted titer and the predicted karyotype. However, this solution has at least the following technical issues: deep learning models usually require large-scale datasets for training and calculations, and processing large-scale datasets may take a long time, thereby influencing the efficiency of automated processing. Therefore, the efficiency of automated processing of test results needs to be improved.

### SUMMARY

### 1. Technical Issues to be Solved

In view of the problem of low efficiency in automated processing of test results in the prior art, the present application provides a titer processing method and system based on deep learning, which are used for the automatic processing of non-Treponema pallidum serological test results. By integrating machine learning and deep learning technologies, including feature extraction, model training, and image convolutional neural networks, the automation processing efficiency of serological test results is improved.

### 2. Technical Solutions

The objectives of the present application are achieved through the following technical solutions.

One aspect of the embodiments of the present specification provides a titer processing method based on deep learning, which is used for the automatic processing of non-treponemal serological test results, comprising: obtaining a first image of non-syphilis treponemal serological test results; judging the clarity of the test image and outputting a second image; applying a convolutional neural network on the second image for detecting circular targets and outputting the center coordinates and radius of the circular targets; segmenting the second image according to the center coordinates and radius of the detected circular targets to extract circular target regions; sorting the extracted circular target regions according to preset sorting rules; using a convolutional neural network to classify the sorted circular target regions and outputting a negative or positive result; calculating a corresponding titer of the negative or positive result.

Further, the step of judging the clarity of the first image and outputting a second image comprises: converting the first image of the input non-treponemal serological test result into a grayscale image; performing edge detection on the grayscale image and calculating the variance and maximum value of the edge image; based on a preset variance threshold and maximum value threshold, judging whether the variance and maximum value meet the requirements of the second image; if the variance is less than the preset variance threshold and the maximum value is less than the preset maximum value threshold, then confirming the first image as the second image and outputting the second image.

Further, the step of applying the convolutional neural network on the second image for detecting circular targets and outputting the center coordinates and radius of the circular targets comprises: obtaining a training set containing non-treponemal serological test images, labeling the circular targets in the training set, and obtaining a labeled data set; using the convolutional neural network as the detection model, freezing the feature extraction layer of the detection model, and modifying the output of the detection model as the center coordinates and radius; using the labeled data set to train the detection model; using the trained detection model to predict the input second image, and outputting the center coordinates and radius of the circular targets.

Further, the step of segmenting the second image according to the center coordinates and radius of the detected circular targets to extract circular target regions comprises: drawing a corresponding circular region according to the center coordinates and radius of the circular target; using binarization processing, setting the circular region as foreground pixel values M1, and setting other regions as background pixel values M2, obtaining a binarized image; performing morphological processing on the binarized image to remove noise regions to obtain a refined binarized image; generating a corresponding mask according to the refined binarized image; subjecting the mask to a bitwise AND operation with the original second image to extract the circular target region.

Further, the step of sorting the extracted circular target regions according to preset sorting rules comprises: calculating the center coordinates of each circular target region detected and segmented; judging the rows where the circular targets are located according to the longitudinal difference between the center coordinates of adjacent circular target regions; comparing the lateral difference between the center coordinates of circular target regions in the same row to determine the columns of the circular targets in each row; generating a sorting index of the circular target regions according to row order and according to column order in the same row; rearranging the order of the circular target regions according to the sorting index.

Further, the step of using a convolutional neural network for classifying the sorted circular target regions and outputting the negative or positive result comprises: obtaining a training set containing the sorted circular target regions and labeling the negative and positive labels; using a pre-trained lightweight convolutional neural network as a classification model; freezing the feature extraction layer parameters of the classification model, and modifying the fully connected layer output of the classification model to be a binary classification, representing the negative and positive results; using the labeled data set to fine-tune the classification model and optimize the model parameters; evaluating the fine-tuned classification model and calculating a prediction probability pi; applying the trained classification model to predict the sorted circular target regions and outputting the corresponding negative and positive results.

Further, the predicted probability pi is calculated using the following formula:

$${p}_{i} = \frac{exp ( {z}_{i} / T )}{\sum j \times exp ( {z}_{j} / T )}$$

Wherein, zᵢ and zⱼ are the i^{th} and j^{th} elements in the input vector z, respectively; j is the index variable iterating all categories in the summation operation; T is the temperature parameter.

Further, the step of calculating the titer corresponding to the negative and positive results comprises: inputting the negative or positive result of each circular target region; setting counters of the number of negative circular targets N1, the number of weakly positive circular targets N2, and the number of strongly positive circular targets N3 of each sample; iterating the negative and positive results of each input circular target region, and updating the corresponding counters according to the negative and positive results; calculating the total number of circular targets N for each sample, the total number of circular targets being the sum of each counter; judging the counter values, determining whether the sample is negative, weakly positive, or strongly positive according to interpretation standards of the non-treponemal serological test, and calculating the corresponding titer; outputting the judged negative and positive results and the calculated titer.

Further, when the number of negative circular targets N1 is equal to the total number of circular targets N, then the non-treponemal serological test result is judged to be negative, and the titer is Y1; when the number of negative circular targets N1 is not equal to zero and the number of strongly positive circular targets N3 is equal to zero, then the non-treponemal serological test result is judged to be weakly positive, and the titer is the number of weakly positive circular targets N2 divided by the total number of circular targets N; when the number of strongly positive circular targets N3 is not equal to zero, then the non-treponemal serological test result is judged to be strongly positive, and the titer is Y2.

Another aspect of the embodiments of the present specification further provides a titer processing system based on deep learning, used for automatic processing of non-treponemal serological test results, comprising: an image clarity judgment module, used for judging the clarity of an input non-treponemal serological test image; a circular target detection module, using a pre-trained convolutional neural network for detecting circular targets in a clear image; a circular target segmentation module, used for segmenting the clear image according to the detection results and extracting circular target regions; a circular target sorting module, used for sorting the extracted circular target regions; a circular target classification module, using a pre-trained convolutional neural network for classifying the sorted circular target regions and outputting negative and positive results; a titer calculation module, used for calculating a corresponding titer according to the negative and positive results.

### 3. Advantageous Results

Compared with the prior art, the advantages of the present application are:
(1) Target detection models such as YOLOv5 (You Only Look Once version 5) are used for realizing the automatic positioning of circular areas, image processing methods are applied for automatically segmenting and extracting circular targets, and convolutional neural networks assist with completing target classification, which improve automation processing efficiency;
(2) Laplacian operators extract clear images to improve detection accuracy, image processing is used to ensure segmentation effects, and pre-trained networks are fine-tuned to improve classification accuracy, thereby improving efficiency while ensuring the accuracy of automated processing;
(3) The classification results of each target are counted, the number of targets of different types is calculated according to rules, and the titer is quantitatively calculated in combination with test standards. The classification results are automatically converted into quantitative indicators, providing a basis for subsequent quantitative analysis and evaluation, thereby improving automation processing efficiency.

### BRIEF DESCRIPTION OF THE DRAWINGS

FIG. 1 is a schematic diagram of a titer processing method based on deep learning of the present application;

FIG. 2 is a schematic diagram of a negative/positive determination process of the present application;

FIG. 3 is a schematic diagram of a titration prediction process of the present application.

### DEFINITIONS OF TERMS

Non-treponemal serological test: Once Treponema pallidum infects the human body, the body rapidly mounts an immune response against the lipid substances released from both the damaged host cells and the surface of Treponema pallidum cells, producing anti-lipid antigen antibodies (also called reagin) within 3 to 4 weeks. These antibodies are primarily a mixture of IgG and IgM antibodies. The non-treponemal test is a flocculation agglutination test that uses cardiolipin, lecithin, and cholesterol as antigens. Reagin reacts with cardiolipin to form an antigen-antibody reaction, lecithin enhances the antigenicity of cardiolipin, and cholesterol increases the sensitivity of the antigen. Cardiolipin and lecithin form a colloidal solution in water, while cholesterol forms crystals in water. When the antigen and antibody (reagin) are mixed and reacted, the products attach to the surrounding colloidal particles, forming a hydrophobic membrane. Due to shaking and collisions, the particles adhere to each other, leading to visible particle agglutination and precipitation, which indicates a positive reaction. If the serum does not contain Treponema pallidum, the albumin concentration in the body fluid will be higher than the globulin concentration, and albumin provides a protective hydrophilic membrane around the colloidal particles. Even with the same shaking and collisions, since no immunoglobulin is attached to the antigen particles, no large particles are formed, and no visible agglutination or precipitation occurs, indicating a negative reaction. RPR and TRUST are examples of this type of test. They use the same antigen components and have similar sensitivity and specificity.

First image: The first image refers to the original digital image of the test result obtained from non-treponemal serological test equipment, without any processing. The first image contains all the information from the non-treponemal serological test, but it may have uneven lighting, blurriness, or other quality issues, making direct analysis and interpretation difficult.

Second image: The second image is the image suitable for further analysis obtained after the first image has undergone clarity judgment and processing. Compared to the first image, the second image retains the original information while improving image quality, such as clearer edges and moderate contrast, facilitating subsequent circular target detection and other operations.

Negativity and positivity: Negativity and positivity are the qualitative determination of the non-treponemal serological test results, indicating whether non-treponemal antibodies are present in the serum sample to be tested. Negativity indicates the absence of non-treponemal antibodies, while positivity indicates the presence. The test often determines the overall negativity or positivity of the serum to be tested based on the negativity or positivity of multiple sample wells (i.e., circular targets).

titer: The titer is a quantitative indicator of the non-treponemal serological test results, representing the assay or concentration of non-treponemal antibodies in the serum to be tested. The titer is generally determined by dilution, where the serum to be tested is serially diluted before testing, and the titer is determined based on the negativity and positivity of the sample wells at different dilution levels. Accurate determination of the titer is of great significance for disease diagnosis and treatment monitoring.

Edge detection: Edge detection is a fundamental operation in image processing and computer vision, used for identifying areas in an image where pixel values change sharply. These areas often correspond to important features such as object edges and contours. The purpose of edge detection is to extract edge information from an image, providing a foundation for subsequent tasks such as object recognition, segmentation, and feature extraction. Adoptable edge detection algorithms include Canny edge detection, Sobel edge detection, and Laplacian edge detection.

Binarization processing: Binarization processing is the process of converting a grayscale image into a black-and-white image containing only two pixel values. By setting a threshold value, pixels with values greater than or equal to the threshold value are set to white (usually assigned a value of 255), while pixels with values less than the threshold value are set to black (usually assigned a value of 0). Binarization processing can simplify the image into foreground and background regions, simplifying subsequent tasks such as object extraction and feature recognition.

Foreground pixel value: The foreground pixel value refers to the pixel value representing the region of interest or target in a binarized image. In the present application, the pixel values within the circular target areas are set as the foreground pixel value M1, indicating that these pixels belong to the region to be extracted. The foreground pixel value is usually assigned a relatively large positive integer value, such as 255.

Background pixel value: The background pixel value refers to the pixel value representing non-target areas or background in a binarized image. In the present application, the pixel values outside the circular target area are set as the background pixel value M2, indicating that these pixels do not belong to the region to be extracted. The background pixel value is usually assigned a relatively small non-negative integer value, such as 0.

Morphological processing: Morphological processing is an image processing method based on morphological operations. Operations performed on an image such as erosion, dilation, opening, or closing can change the shape of target regions, remove noise, and fill holes. Common morphological operations include: dilation, which expands white regions or foreground regions in an image; erosion, which shrinks white regions or foreground regions and removes small noise points; opening, which consists of erosion followed by dilation and is used to remove small noise points and break fine connections between targets; and closing, which consists of dilation followed by erosion and is used to fill small holes within targets and connect small breaks in targets.

Noise regions: The noise regions refer to interference information or irrelevant pixels in an image, often appearing as random dots, patches, or textures. Noise regions may result from imaging equipment defects, environmental interference, and other factors. In the present application, the binarized image may contain small noise regions outside the circular target areas, which need to be removed through morphological processing to avoid interference with subsequent processing.

Mask: A mask is a binarized image of the same size as the original image, used for indicating the positions of the regions of interest. In the mask image, the pixel value for the regions of interest (such as the target) is set to 1 or 255, while the pixel value for other regions is set to 0. In the present application, a mask is generated based on the refined binarized image, wherein the pixel value inside the circular target areas is 1, and the pixel value outside the areas is 0. By performing a bitwise AND operation between the mask and the original image, the regions of interest can be extracted while background information is masked.

### DETAILED DESCRIPTION OF EMBODIMENTS

The present application is described in detail below in conjunction with the accompanying drawings and specific embodiments.

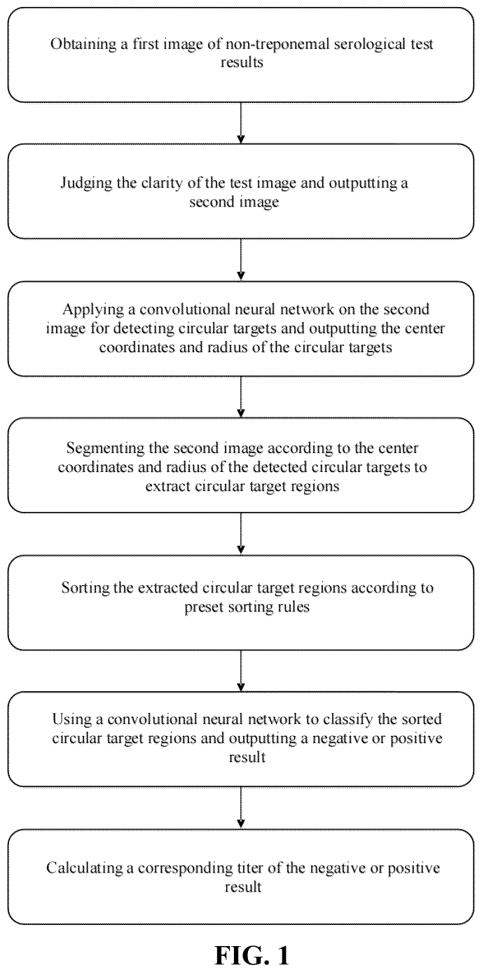

FIG. 1 is a schematic flowchart of a titer processing method based on deep learning of the present application. As shown in FIG. 1, the titer processing method based on deep learning comprises the following technical steps:

Obtain a first image of non-treponemal serology; this image is an initial input image for serological testing. Wherein, non-treponemal serology is a serological test method used for detecting syphilis (Treponema pallidum infection). Syphilis is a sexually transmitted disease caused by infection with Treponema pallidum. Non-treponemal serological tests can be used to assist in determining whether a patient is infected with syphilis and the severity of the infection. Such tests include RPR (Rapid Plasma Reagin), TRUST (Toluidine Red Unheated Serum Test), and VDRL (Venereal Disease Research Laboratory Test). At present, RPR and TRUST are the two main methods used in Mainland China. In the present application, obtaining the first image of the non-treponemal serological test (RPR and TRUST) result means obtaining the initial input image for the serological test. This image is usually a recorded image of a patient's serum sample during the experimental process for result interpretation, used for subsequent automated processing and titer calculation.

Use a pre-trained convolutional neural network model YOLOv5 to perform circular target detection on a secondary image and output the center coordinates and radius of each detected circular target. This step achieves automated and efficient target localization through deep learning; wherein, YOLOv5 (You Only Look Once v5) is a target detection model based on convolutional neural networks, used for achieving efficient object localization and recognition. In the above technical solution, the pre-trained YOLOv5 model is used for circular target detection on a second image to improve the automation efficiency and accuracy of serological test result processing.

YOLOv5 is one of the models in the YOLO series, which uses a target detection method called “one-stage.” Compared with the traditional “two-stage” method, YOLOv5 can achieve target detection and localization simultaneously in a single forward propagation process, significantly improving efficiency. It segments an image into multiple grid units and predicts a bounding box and category information of the target within each unit. In this way, YOLOv5 can quickly and accurately locate and identify circular targets in the image. In this solution, YOLOv5 is used as the backbone of the circular target detection model, with some parameters frozen and the output layer modified so that it can output the center coordinates and radius of each detected circular target. By pre-training the YOLOv5 model and training it with a labeled dataset, the model can automatically detect and extract circular target information from the second image. This can reduce the workload of operators and realize automated interpretation of serological test results. The advantage of using YOLOv5 lies in its efficiency and accuracy. It can detect multiple targets in an image within a short time while providing target location and size information. Compared with traditional methods that rely on manually designed features and multi-stage processes, YOLOv5 can perform target detection quickly and perform excellently in real-time scenarios. This further improves the automation efficiency of serological test result processing and provides accurate input for subsequent steps such as target segmentation and classification.

Use the pre-trained convolutional neural network model YOLOv5 to perform circular target detection on the secondary image and output the center coordinates and radius of each detected circular target. This step achieves automated and efficient target localization through deep learning; based on the detection results, image processing methods (such as binarization and morphology) are used for automatically segmenting and extracting each circular target region, for preparing the classification and calculation of input regions; sort the extracted circular target regions according to coordinate relationships to ensure that the target region order of the same sample remains consistent. This step ensures the standardization of subsequent classification input; use a convolutional neural network to classify the sorted circular target regions and output a positive or negative result. By training on circular targets in serological tests, this step can classify each circular target and determine whether the result is negative or positive; calculate the corresponding titer for negative and positive results. Based on the classification results and combining the titer calculation rules or algorithms, the corresponding titer is calculated. This titer provides a quantitative analysis of serological test results, which can be used for further judgment and evaluation.

In summary:

Automated target localization: Use the pre-trained convolutional neural network model YOLOv5 to perform circular target detection on images and output the center coordinates and radius of the targets. This automates the target localization process, eliminating the need for manual intervention and significantly improving efficiency and accuracy.

Automated target segmentation: Use image processing methods (such as binarization and morphology) to automatically segment and extract each circular target region. This isolates each target region, preparing it for subsequent classification and calculation.

Standardized classification input: By sorting the extracted circular target regions according to coordinate relationships, the consistency of the target region order of the same sample is ensured. This classification input increases standardization, allowing subsequent classification algorithms to accurately determine the nature of each target.

Accurate classification and interpretation: Use a convolutional neural network to classify the sorted circular target regions and output a negative or positive result. By training the network to adapt to the characteristics of serological tests, the present application can accurately classify each target and determine whether the result is negative or positive.

Quantitative analysis of titers: Based on the classification results and combining the calculation rules or algorithms of titers, the corresponding titer is calculated. This titer provides a quantitative analysis of serological test results, making the results more measurable and meaningful for guidance.

Through the above technical effects, the present application enables the automated processing of titers from the original serological test image, significantly improving efficiency and accuracy. It reduces the need for manual intervention, minimizes the risk of human error, and provides doctors and researchers with a fast, reliable, and quantitative serological test result analysis tool.

Wherein, the step of determining the clarity of the test image and outputting the second image comprises: convert the input first image of non-treponemal serology into a grayscale image. Converting a color image into a grayscale image simplifies subsequent processing steps, focusing only on grayscale information in the image. Use the Laplacian operator to perform edge detection on the grayscale image and calculate the variance and maximum value of an edge image. The Laplacian operator is a commonly used edge detection algorithm that extracts edge information from an image. In this step, the Laplacian operator is applied to process the grayscale image, obtain the edge image, and calculate the variance and maximum value of the edge image.

Specifically, the first image of serology is converted into a grayscale image. Converting a color image into a grayscale image can reduce the complexity of subsequent processing steps. In object detection and image processing tasks, grayscale images are usually sufficient to provide useful information without considering the impact of color channels on the results. This simplification helps improve processing speed and reduces the use of computational resources. The targets in serological test images usually have specific grayscale distributions and texture features. By converting into a grayscale image, changes in grayscale intensity in the image can be focused on without dealing with color information. This can enhance the contrast of targets in the image, making subsequent image analysis and processing more accurate and reliable. The processing and feature extraction of grayscale images can serve as an automated preprocessing step, providing accurate input for subsequent image analysis and target detection. When using the pre-trained convolutional neural network model YOLOv5 for circular target detection, the processing of grayscale images can provide better image quality and feature representation, thereby improving the accuracy and performance of target detection.

Specifically, the Laplacian operator is a commonly used edge detection algorithm that can extract edge information from images. In serological testing, the circular edges of targets are often one of the important features. By applying the Laplacian operator for edge detection on grayscale images, the edge structures of targets can be emphasized, providing an accurate basis for subsequent target localization and measurement.

More specifically, convert an input non-treponemal serological image I into a grayscale image Ig; apply the Laplacian operator to the grayscale image Ig for edge detection to obtain an edge image E; the Laplacian operator template is defined as:

$$\vert \begin{matrix}0 & - 1 & 0 \\ - 1 & 4 & - 1 \\ 0 & - 1 & 0\end{matrix} \vert$$

Calculate the variance of the edge image E: the variance formula is:

$${σ}^{2} = ( 1 / N ) ⋆ \sum {( {E}_{i} - μ )}^{2}$$

Wherein N is the total number of pixels in the edge image, Eᵢ is the i^{th} pixel value, and μ is the mean of all pixels in the edge image.

Calculate the maximum pixel value max_value in the edge image E. Use the calculated variance and maximum pixel value max_value as the edge features of the image to subsequently determine whether the image clarity meets requirements. Compare the calculated variance and maximum value with a preset variance threshold and a maximum value threshold to determine whether to retain or filter the image. This technical solution utilizes the Laplacian operator for edge extraction, and by determining clarity based on feature values, effectively filters blurred images, and meets automated processing requirements. According to the preset variance threshold and the maximum value threshold, whether the variance and maximum value meet the requirements of the second image is determined. Based on specific needs, the variance threshold and maximum value threshold are set to determine whether the image clarity meets the requirements. By comparing with preset thresholds, the degree of clarity of the image can be determined; wherein, the technical solution for setting the variance threshold is similar to that of setting the maximum value threshold, and is not elaborated herein.

Wherein, the technical solution for setting the maximum value threshold:

Collect non-treponemal serological test images with different clarity levels, and perform subjective clarity scoring for each image. The scoring standard ranges from 1 to 5, with higher scores indicating clearer images. Apply the Laplacian operator to each image for edge detection and calculate the maximum pixel value max_value of the edge image. Perform statistical analysis on the subjective scores and maximum pixel values max_value of each image and fit the mapping relationship between the scores and the maximum pixel values. Determine the minimum clarity score threshold, for example, setting a threshold score of 3, and use the corresponding maximum pixel value as the maximum value threshold max_threshold. In practical applications, apply the Laplacian operator to the input image for edge detection and calculate the maximum pixel value max_value. Compare max_value with the maximum value threshold max_threshold; if max_value is greater than or equal to max_threshold, the image clarity is deemed acceptable, otherwise, the image is discarded. This solution for setting the maximum value threshold obtains threshold parameters based on sample data analysis, which can effectively filter blurred images, ensuring the clarity of output images, improving subsequent processing effects, and enhancing interpretation accuracy.

If the variance is smaller than the preset variance threshold and the maximum value is smaller than the preset maximum value threshold, then confirm the first image as the second image and output the second image. When the clarity requirements are met, the original image is output as the second image. The present application can determine the clarity of the input non-treponemal serological image and output the second image. Such clarity determination can help improve the automation processing efficiency of serological test results.

Specifically, in the present embodiment, the input image is first converted into the grayscale image, and then the Laplacian operator is applied for edge detection to calculate the variance Var and maximum value Max of the edge image Img. The variance Var and maximum value Max are compared with preset thresholds A and B. If the conditions Var<A and Max<B are met, the image is determined as a clear image, and the next step is executed; if the conditions Var<A or Max<B are not met, an error message is returned, requiring the input of an image that meets the clarity requirements. When the input image meets the clarity requirements, the input image is confirmed as the second image, and the second image is output as the serological test detection result. By combining image processing technology with clarity determination criteria, automated processing of serological test detection results is achieved. Determination is based on quantifiable parameters, which reduces the influence of subjective errors. Automated processing reduces manual operations and improves efficiency.

Wherein, the technical steps of using a convolutional neural network to detect circular targets in the second image and output the center coordinates and radius of the circular targets, thereby improving the automation processing efficiency of serological test detection results, comprises:

Obtain a training set containing non-treponemal serological test images and label the circular targets in the training set to obtain a labeled dataset; label the circular targets in the training set to obtain a labeled dataset. The labeled dataset comprises boundary box information of circular targets or pixel-level mask information. By selecting YOLOv5s as the detection model network backbone, compared with larger models, the YOLOv5s model is smaller in size, requires fewer computational resources, has a faster prediction speed, and is more suitable for real-time automated processing systems with high requirements. This can reduce the time required for a single judgment and improve system throughput. By freezing the feature extraction layer parameters of the network backbone and directly utilizing the pre-trained semantic feature extraction capability of the YOLOv5 model, training a feature extractor from scratch is avoided, reducing training costs and time, and improving model fine-tuning efficiency. Modifying the model output head in the format of center coordinates and radius can directly adapt to the characteristics of circular targets, eliminating the need for post-processing to adjust the output format, simplifying the process, reducing operations, and improving judgment speed. By using YOLOv5s as the detection model network backbone, combined with pre-trained parameters in this field, detection results are excellent, missed detections and false detections can be reduced, the proportion of failed re-detection samples is lowered, and efficiency degradation is prevented.

In summary, this design fully utilizes the computational advantages of the models of the YOLOv5 series. By freezing parameters, adjusting output, simplifying processes, and other methods, the automation processing efficiency of circular target detection in non-treponemal serological images is effectively improved while ensuring detection accuracy.

Use the labeled dataset to train the detection model; by inputting the training set and label information into the model to perform backpropagation and parameter updates, the model is caused to learn the features and location information of circular targets, which iteratively trains the model and optimizes model parameters to improve the detection accuracy and generalization ability of circular targets.

Use the trained detection model to predict the input second image and output the center coordinates and radius of circular targets; extract the center coordinates and radius information of the circular targets through the prediction results of the detection model.

Through convolutional neural network training and prediction, automatic detection of circular targets in the second image is achieved, and the center coordinates and radius are output. Based on deep learning model training, accuracy and generalization ability are improved, which is suitable for circular target detection tasks of different shapes and sizes.

Specifically, in the present embodiment, 1000 syphilis images are collected, of which 600 are TRUST samples and 400 are RPR samples; the images are divided into a training set, a validation set, and a test set in a ratio of 6:2:2; in the training set, circular well positions in each image are labeled using a script, and the label results are manually adjusted to generate a labeled dataset.

For model training and evaluation, YOLOv5s is adopted as the selected detection model, serving as the basis of the present application. The YOLOv5s model is trained using the labeled dataset, and iterative optimization of model parameters improves the detection accuracy and generalization ability of circular well positions. The validation set during the training process is evaluated to compute model performance metrics such as accuracy, recall, and F1-score on the validation set. Model testing and performance evaluation use the test set to evaluate the performance of the trained model, calculating the accuracy and other evaluation metrics of the model. The speed of the model is tested, and the time taken to detect all circular well positions in a single image is recorded. In the present embodiment, detecting all circles in one image takes approximately 0.1 second. The performance and robustness of the model are evaluated on a large-scale test set.

Specifically, YOLOv5s adopts a small network structure with fewer parameters and computational complexity. It employs a lightweight feature extraction network to reduce model complexity, thereby decreasing computational load during inference. YOLOv5s utilizes the PyTorch deep learning framework, leveraging its underlying optimization features. PyTorch has efficient map computation capabilities, enabling high inference speed. YOLOv5s employs a training strategy called Self-train, which enhances the target perception ability of the model by pre-training with a larger-scale source dataset. This allows the model to achieve better detection performance in the test phase after lightweight fine-tuning on the training set. YOLOv5s uses a feature cascade strategy, fusing low-level and high-level features to assist in improving detection capability for targets of different scales. This feature cascade design enhances the recall rate and accuracy of the model. By adopting characteristics such as a lightweight network structure, bottom-layer optimization, target-sensing pre-training, and feature cascade, YOLOv5s improves the efficiency of object detection. This ensures that YOLOv5s remains competitive in inference speed while maintaining high detection accuracy.

Using the YOLOv5s model as the circular well detector, in the present embodiment, after training and optimization, the detection accuracy of the present application reaches 99.6%, effectively detecting circular wells in images. The present application, through an optimized model architecture and algorithm, achieves the detection of all circles in one image within 0.1 second, making it suitable for real-time monitoring scenarios. The YOLOv5s model, compared to other models, has a smaller number of parameters, facilitating model deployment and inference. This method replaces traditional image processing methods, improving the detection accuracy and robustness of circular well positions while maintaining high speed and deployment convenience, thereby enhancing the automation processing efficiency of serological test detection result processing.

Wherein, based on the center coordinates and radius of the detected circular targets, the second image is segmented to extract circular target regions to achieve automated processing of serological test detection results. The technical steps comprise: draw the corresponding circular regions according to the center coordinates and radius of the detected circular targets. The circular region is designated as a region of interest for extracting the circular target. Binarization processing is performed on the second image, setting the circular region as the foreground and other regions as the background. Threshold segmentation or other binarization methods can be used to set the pixel values of the circular region as M1 and set the pixel values of the non-circular region as M2, obtaining a binarized image. The circular region is drawn according to the detection results to avoid recalculating the target positions, improving segmentation efficiency. In the present embodiment, M1 is set to 1, and M2 is set to 0. Morphological processing is performed on the binarized image to remove noise and smooth boundaries, obtaining a refined binarized image. Morphological operations such as erosion, dilation, opening, or closing operations can be used to adjust the binarized image structure, making the circular target region more accurate. Binarization processing simplifies the problem into extracting the foreground and background, reducing segmentation difficulty and improving speed. By setting a reasonable threshold, the circular target region can be effectively extracted.

A corresponding mask is generated based on the refined binarized image. The foreground region of the refined binarized image is extracted to form a mask for bitwise AND operation with the original image. Wherein, the corresponding mask is generated based on the refined binarized image by: first, loading the refined binarized image into the program or library. Image processing algorithms such as threshold segmentation and connected region analysis extract the foreground region in the binarized image. The foreground region represents the target or region of interest, usually marked in white (value 1). A blank image of the same size as the original image is created, filling the foreground region with white (value 255) while keeping the background region black (value 0). This generates a mask image where white pixels represent the foreground region and black pixels represent the background region.

Specifically, loading the refined binarized image directly utilizes image processing results, avoiding reprocessing and improving efficiency; using threshold segmentation and other algorithms to extract the foreground region accurately acquires the target region without designing new algorithms; creating a blank image as a mask, matching the original image in size, meets subsequent operation needs; filling the extracted foreground region with 255-pixel values represents the target region. The background is filled with 0-pixel values. This generated mask directly differentiates foreground and background without complex design. The 255-pixel value in the mask accurately marks the target region information in the original image. Subsequent bitwise AND operations with the original image directly extract the region of interest. This mask generation design is simple and effective, using existing techniques for accurate extraction, avoiding redundant processing, and improving process efficiency. Quickly generating masks while ensuring extraction effectiveness meets the processing speed and quality requirements of automated processing. In summary, this technical setup fully considers the efficiency and effectiveness requirements of automated processing systems, utilizing simple and efficient technical means to generate accurate masks, and providing support for subsequent improvement of judgment efficiency.

The mask is subjected to a bitwise AND operation with the original second image to extract the circular target region. The result of the bitwise AND operation retains pixel values corresponding to the foreground region in the mask while setting other regions to 0, thereby extracting the circular target image region. Wherein, the bitwise AND operation refers to: performing a bitwise AND operation between the original image and the generated mask. The bitwise AND operation keeps the pixels in the original image that are white at the corresponding positions in the mask while setting pixels of other positions to black. This ensures that only parts of the original image corresponding to the foreground region are retained while other regions are removed. Image processing libraries (such as OpenCV) or image processing functions in programming languages can be used for implementation.

Through steps such as drawing circular regions, binarization processing, morphological processing, and bitwise AND operation, effective extraction of circular target regions in the second image is achieved. The morphological processing step can effectively remove noise and smooth boundaries, improving the accuracy of circular target regions. The extracted region is automatically adjusted according to the center coordinates and radius of the detected circular target, adapting to circular targets of different sizes and positions. It is used in serological testing to extract circular target regions, such as circular well positions in syphilis images. The present application enables effective extraction of circular target regions from the second image.

Specifically, in the present embodiment, circular targets are sequentially arranged and matched with input circle indices based on the center coordinates and radius returned by YOLOv5s detection to improve the automation processing efficiency of serological test results. The detailed technical solution is as follows: the number of rows is determined by comparing the radius differences of adjacent circles. The code checks the radius difference between every two adjacent circles; if this difference exceeds a preset value (e.g., self.h_r), the row count is increased.

Specifically, the setting of the preset value (also known as the threshold) is determined based on specific application scenarios and needs. It dictates how much of a radius difference is considered significant enough to increase the row count.

The process of specifically setting the preset value may require some experimentation and adjustment in order to achieve the best results. The following are some possible methods to determine the preset value:

Observing data: First, carefully observe the data to understand the differences between the radii of the circles. Through observation, a rough judgment of the typical range of differences between radii can be made and used as an initial estimate for the preset value.

Statistical analysis: If a large number of circular samples is available, statistical analysis can be conducted on the differences in circle radii. Calculate statistical indicators such as the mean and standard deviation of the radii differences, and select a suitable preset value as a threshold based on the distribution of the data.

Trial-and-error method: Different preset values can be tested, and the accuracy and alignment of the results with expectations can be observed. Adjust based on the results until the best preset value is found.

Experimental verification: According to specific application scenarios, some sample data can be selected for experimental verification. The size of the preset value can be determined based on the experimental results to meet actual needs.

By dividing the total number of circles by the number of rows, the number of circles per row, i.e., the number of columns, is determined. To ensure the correct order of the circles, the code groups the circles by rows and sorts them within each group according to the x-coordinate of the center (i.e., column). This ensures that the circles are not only ordered between rows but also within the same row. All sorted rows are combined into a result array to ensure that all circles are arranged in the expected order. In this way, the sequential arrangement of circular targets and the matching of input circle indices are completed. By determining the number of rows based on the differences in circle radii, reordering by rows, and sorting circles according to the center x-coordinates of the circles, the sequential arrangement of circular targets and the matching of input circle indices are achieved. Through an automated algorithm, the arrangement and index matching of circular targets can be completed without manual intervention. The optimized arrangement and matching method can improve the automation processing efficiency of serological test results, reducing manual operation time and errors. It is used for the sequential arrangement of circular targets and the matching of input circle indices in serological tests, such as the arrangement of circular wells in serological reagent trays. The present application allows for the quick and accurate sorting of circular targets and matching of input circle indices, providing accurate data support for subsequent detection result analysis and processing.

Wherein, the extracted circular target regions are sorted according to a preset sorting rule to improve the automation processing efficiency of serological test results. First, the center coordinate is calculated for each detected and segmented circular target region. By analyzing images or detection results, the center point position of each circular target region is determined. Based on the vertical difference in center coordinates of adjacent circular target regions, the row in which the circular target is located is determined. By comparing the vertical difference in center coordinates of every two adjacent circular target regions, if the difference exceeds a preset threshold, they are considered to be in different rows. Specifically, a target detection algorithm (such as deep learning-based target detection algorithms like YOLO, Faster R-CNN, etc.) is used for detecting circular target regions and obtaining their center coordinates. The detected circular target regions are sorted based on the vertical coordinates of the center coordinates. The sorted circular target regions are iterated, and the vertical difference in center coordinates of every two adjacent circular target regions is compared. If this difference exceeds a preset threshold, they are regarded as being in different rows. By counting the number of times the vertical difference exceeds the preset threshold, the total number of rows is determined.

Within the same row, the horizontal difference in center coordinates of the circular target regions is compared to determine the columns of the circular targets in each row. Based on the horizontal difference, the circular target regions are sorted by columns to ensure that the circular targets in the same row are arranged in horizontal order. Following the row order, each row is sorted first, and then within the same row, the column order is followed to generate a sorting index for the circular target regions. A unique sorting index is generated based on the row and column numbers of each circular target to indicate the sorted order. According to the generated sorting index, the sequence of circular target regions is rearranged. Following the sorting index, the circular target regions are rearranged in the preset order to achieve an orderly arrangement of the target regions.

Through steps such as calculating center coordinates, determining rows and columns, generating sorting indices, and rearranging the sequence, the sorting of circular target regions based on preset sorting rules is achieved. The optimized sorting method can improve the automation processing efficiency of serological detection results and is used for sorting circular target regions in serological detection, such as the arrangement of circular wells in serological reagent trays. The present application allows for the quick and accurate sorting of circular target regions.

The technical solution for circular target region sorting is as follows:
Detect each circular target and obtain its center coordinates (x, y);
Calculate the vertical difference Δy=y₁−y₂| between all target region center points;
Set a vertical difference threshold, such as 50 pixels;
Compare Δy with the threshold. If Δy>the threshold, the two targets are in different rows;
Count the occurrences where the vertical difference Δy>the threshold to get the total number of rows, m;
Within the same row, calculate the horizontal difference Δx=x₁−x₂|;
Sort Δx in ascending order to get the horizontal order of targets in the same row;
Generate row numbers (1 to m) and column numbers (1 to n) for each target;
Sort by row numbers in ascending order, and within the same row, sort by column numbers in ascending order to generate a unique sorting index;
Adjust the sequence of each target region according to the sorting index;
The final result is a collection of target regions sorted by row and column for subsequent processing.

The present application fully utilizes center coordinate information, sets reasonable threshold parameters, and rearranges target order through sorting indices to achieve effective sorting. This sorting process has low computational complexity, is easy to implement, and can improve the efficiency of automated processing workflows.

Specifically, in the present embodiment, based on the HSV color space and threshold processing technology, HSV represents Hue, Saturation, and Value. The HSV color space is also called the HSB color space, which is a commonly used color representation method. Wherein: Hue represents the type of color, such as red, yellow, etc., with a range of 0-360°. Saturation represents the vividness of the color, with a range of 0-100%. The higher the value, the more vivid the color. Value represents the brightness of the color, with a range of 0-100%. The higher the value, the brighter the color. Compared with the RGB color space, the HSV space is more in line with the human subjective visual perception of colors, so it is more widely used in the field of image processing and computer vision. Converting an RGB image to the HSV space allows convenient analysis and processing of the image based on hue, saturation, and other features. By detecting the blue region, finding contours and calculating the enclosing circle, and performing mask merging operations, the automatic detection and extraction of circular regions are achieved. The detailed technical solution is as follows: convert the original image from the RGB color space to the HSV color space. The HSV color space consists of three components: Hue, Saturation, and Value, which are more suitable for color analysis and processing. Perform threshold processing in the HSV image according to a predefined blue threshold range to filter out blue regions. By adjusting the threshold range, the detection of blue regions can be optimized according to actual needs. Use the cv2.findContours(X) function to find contours in the threshold-processed image. This function can detect all contours in the image and return the set of points for each contour. From the found contours, find the largest contour. The largest contour usually corresponds to the target region, which is the circular region here. Calculate the enclosing circle of the largest contour. Using the cv2.minEnclosingCircle(X) function, the enclosing circle center coordinates and radius information of the largest contour can be obtained. Create a mask image of the same size as the original image, set the pixels inside the circular region to white (255), and set the rest of the regions to black (0). Then, use a bitwise AND operation to merge the original image with the mask, retaining only the pixels inside the circular region. Through HSV color space conversion, blue region detection, contour searching, enclosing circle calculation, and mask merging steps, the automatic detection and extraction of circular regions are achieved. The method based on the HSV color space and contour extraction can accurately detect and extract circular regions.

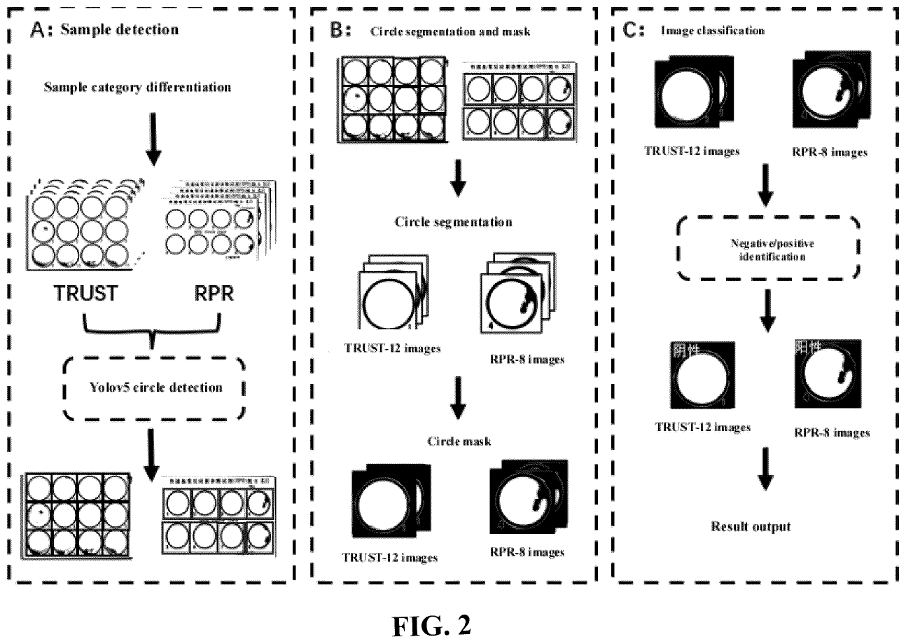

FIG. 2 is a schematic diagram of a negative/positive determination process of the present application. As shown in FIG. 2, a convolutional neural network is used for classifying the sorted circular target regions, and the steps for outputting the negative/positive result comprise:

Obtain a training set containing sorted circular target regions, and annotate negative/positive labels. Ensure that the dataset contains a sufficient number of samples representing negative/positive results for effective classification training. Select a pre-trained lightweight convolutional neural network as the classification model. Pre-trained models usually have good feature extraction capabilities and can speed up the entire classification model training process. The technical solution for sorting circular target regions is as described above and will not be repeated. Fine-tune the classification model using the labeled dataset and optimize the model parameters. By training on the labeled dataset, the model can learn to distinguish the feature representations of different negative/positive results. Evaluate the fine-tuned classification model and calculate classification accuracy metrics to determine the performance of the classification model. Cross-validation or an independent test set can be used to evaluate classification accuracy and generalization ability. Apply the trained classification model to the sorted circular target regions for prediction, and output the corresponding negative/positive judgment result. According to the classification model output, each circular target region is determined as negative or positive. By using a convolutional neural network to classify the sorted circular target regions, the automated processing of serological test results is achieved. The convolutional neural network has powerful feature extraction and classification capabilities, enabling accurate determination of negative/positive results in circular target regions.

Using the TRUST and RPR sample syphilis classification models developed based on knowledge distillation methods, the application of these models in improving the automation efficiency of serological test results is explained. The following are the specific steps and technical features:

Knowledge distillation is a model compression technique that transfers the knowledge of a large teacher model to a small student model. Its core idea is to use a teacher model pre-trained on a large dataset to guide student model training, thereby improving the performance of the student model. The present application proposes a classification accuracy metric calculation method based on knowledge distillation to evaluate the fine-tuned classification model and calculate the predicted probability pi. The predicted probability pi is calculated using the following formula:

$${p}_{i} = \frac{exp ( {z}_{i} / T )}{\sum j \times exp ( {z}_{j} / T )}$$

Wherein, zᵢ and zⱼ are the i^{th} and j^{th} elements in the input vector z, respectively; j is the index variable iterating all categories in the summation operation; T is the temperature parameter. The present application, through the idea of knowledge distillation, uses soft labels (predicted probabilities) instead of hard labels to calculate metrics, which can more comprehensively evaluate the classification performance of the model. By adjusting the temperature parameter T, the prediction probability values of different categories can be balanced, further improving the differentiation of the metric. Compared with directly using classification accuracy, this metric can provide more fine-grained information, which helps for analysis and optimization of model performance.

Wherein, the original classification accuracy metric pi is a special case of T=1. The present application can smooth the output probability distribution by adjusting the parameter T, increasing the impact of negative labels on model training, thereby improving model performance on small datasets. Based on the idea of knowledge distillation, the knowledge of the teacher model can be effectively utilized to enhance the performance of the student model on small datasets. By adjusting the temperature T, the output probability distribution can be smoothed, enhancing the model training effect. The present application has low training time requirements, making it suitable for weaker GPU environments.

In the present embodiment, the TRUST sample syphilis classification model:

Dataset collection: 2500 syphilis well images are collected as training data, of which 1200 are positive and 1300 are negative.

Dataset partitioning: The dataset is segmented into a training set, a validation set, and a test set in a 6:2:2 ratio for model training, parameter tuning, and performance evaluation.

Model training and script classification: using a method based on knowledge distillation, under the guidance of the teacher model, script classification is performed on the circles in each image in the training set, categorizing them into negative and positive.

### RPR Sample Syphilis Classification Model:

Dataset collection: 1,000 syphilis well images are collected as training data, of which 450 are positive and 550 are negative.

Dataset partitioning: the dataset is segmented into a training set, a validation set, and a test set in a 6:2:2 ratio for model training, parameter tuning, and performance evaluation.

Model training and script classification: using a method based on knowledge distillation, under the guidance of the teacher model, script classification is performed on the circles in each image in the training set, categorizing them into negative and positive.

Combining the application of technical features, both the TRUST sample syphilis classification model and the RPR sample syphilis classification model adopt a method based on knowledge distillation, which has advantages in improving the automation processing efficiency of serological test detection results. The TRUST sample has a larger data volume and better detection performance, making it more commonly used in clinical testing. By selecting appropriate datasets, training models, and optimization, classification performance and automation processing effects can be improved.

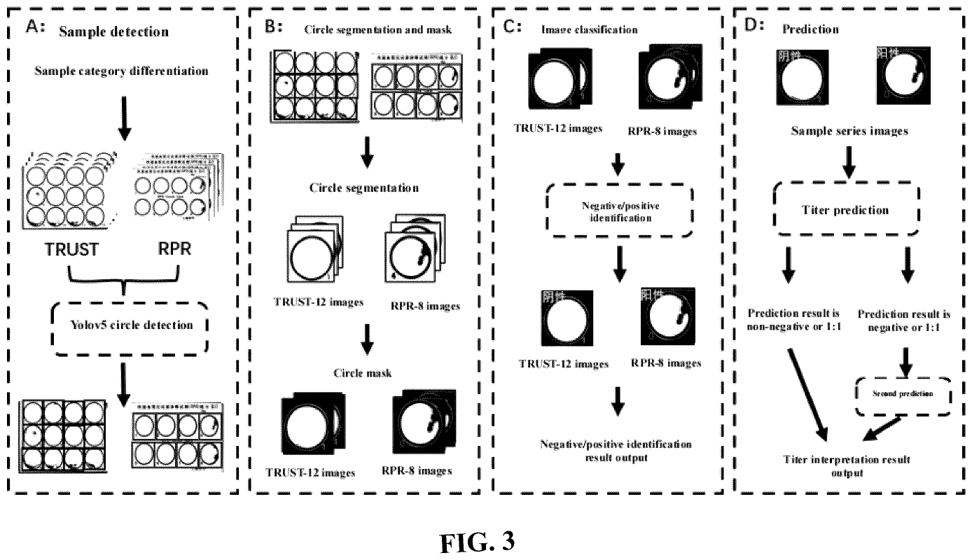

FIG. 3 is a schematic diagram of a titration prediction process of the present application. As shown in FIG. 3, the input original image is detected using the yolov5s object detection model to obtain the number and positions of all circles in the template; based on the detected circle positions, the original image is segmented into 12 or 8 circle regions; index matching is performed to filter out doctor-designated target circles and sort them by position; mask processing is applied to the circle regions to exclude interference factors outside the circles; binarization classification is performed on each circle region to determine whether it is negative or positive; three counters—negative (N1), weakly positive (N2), and strongly positive (N3)—are created, the classification results are iterated, and the corresponding counters are updated; based on the counter values, the titer level is determined, and the predicted titer is output. A secondary verification is performed on negative and 1:1 titer results, and the final prediction result is output.

The present application utilizes counters to count the target quantity and calculates the titer according to interpretation rules. The process is simple and clear. Complex algorithms are avoided to speed up calculations and improve automation processing efficiency. Wherein, by inputting the negative/positive classification results of each circular target region, region extraction and classification are avoided, improving computational efficiency. Multiple counters are set to count different types of targets, simplifying subsequent statistical judgment logic and accelerating computation speed. The iteration results update the counters, replacing manual counting with program control, reducing computation time and improving efficiency. Summing calculates the total number of targets, and the test result type is quickly determined according to preset positive and negative judgment rules. Complex algorithms are avoided to improve judgment speed. The titer is calculated based on a standard equation, and quantitative analysis metrics are directly output based on the input counted results. Given the input, the qualitative and quantitative analyses can be quickly and automatically completed using the counting statistics and judgment rules, avoiding manual interpretation and shortening judgment time. The process is simple and reliable, easy to optimize and parallelize, which helps further enhance throughput and improve the overall efficiency of the automated processing system. This module design fully considers efficiency requirements, simplifies the process by setting appropriate conditions, and achieves rapid and accurate negative and positive judgment and titer calculation, thereby improving the automation processing efficiency of non-treponemal syphilis serological test results.

Specifically, when the number of negative circular targets N1 equals the total number of circular targets N, then the non-treponemal syphilis serological test result is determined to be negative, and the titer is Y1; when N1 equals N, it is determined as negative. When all targets are determined to be negative, it indicates that the sample does not contain antibodies overall and is a negative result, and the titer is set to Y1 to indicate negativity. In the present embodiment, Y1 is set to 0. The automated target detection and localization method (such as using the YOLOv5 model) can efficiently process multiple targets in an image while obtaining the center coordinates and radius of each target. This allows a large number of serological images to be processed in a short time and to be rapidly and accurately determined as negative. The titer Y1 is set to 0 to represent a negative result. This unified result representation method simplifies result interpretation and subsequent data analysis steps. Furthermore, it also provides a standardized result output method, facilitating further data processing and statistical analysis.

When the number of negative circular targets N1 is not equal to zero and the number of strongly positive circular targets N3 is equal to zero, then the non-treponemal syphilis serological test result is determined to be weakly positive, and the titer is the number of weakly positive circular targets N2 divided by the total number of circular targets N; when N1 is not equal to 0 and N3 is equal to 0, it is determined as weakly positive. This means that a negative reaction exists but there is no strongly positive reaction, which is classified as weakly positive according to standards. The titer is N2/N, which is the proportion of weakly positive targets in the total, indicating the degree of weak positivity. Through the titer N2/N, the degree of weak positivity in serological test results can be quantified. In this way, doctors or researchers can further analyze and interpret results based on the titer size to better assess the infection level or disease status of the patient. The determination and titer of weakly positive results provide more detailed information beyond a simple binary positive or negative classification. This type of quantitative result helps provide more comprehensive data, supporting doctors in making more accurate decisions in clinical diagnosis or research analysis.

When the number of strongly positive circular targets N3 is not equal to zero, then the non-treponemal syphilis serological test result is determined to be strongly positive, and the titer is Y2. When N3 is not equal to 0, it is determined as strongly positive. The presence of a strongly positive reaction determines positivity, without distinguishing the degree of strong positivity, and the titer is uniformly set to Y2 to represent the strongest positivity. In the present embodiment, Y2 is set to 1. When the number of strongly positive circular targets is not zero, then a strongly positive reaction exists, and the serological test result can be accurately determined as positive. This prevents misclassifying strongly positive results as weakly positive, negative, or other categories. A fixed titer Y2 is used as the unified representation of strong positivity without distinguishing degrees of strong positivity. This consistent result representation method can simplify result interpretation and subsequent data processing steps, providing a standardized positive result output method that facilitates further data analysis and statistical processing. Setting the titration value to the strongest positivity identifier (1 in the present embodiment) can more clearly indicate the intensity of the strongly positive reaction.

Specifically, in the present embodiment, in the syphilis classification process, prediction is performed using the detect_batch_deit function. During the initialization step, two class names are defined, namely yin and yang. Then, for each image file under the input image path, a mapping is created to map file names to their corresponding image contents. The images are sorted based on the incoming sort_method parameter. When sort_method is 1, sorting is performed based on the last digit in the file name; when sort_method is 2, sorting is performed based on the first digit in the file name. Firstly, whether save_path exists is checked, and if not, the path is created. Then, all existing files in the path are deleted to make space for new output results. The image processing loop:

The following operations are performed for each image in image_maps:

The model is used to perform inference on the image to obtain a prediction result. The detect_batch_deit function is used for performing inference operations. A prediction label is added to a negative_positive_result list for recording the prediction result of each image. The prediction result is visualized on the image and saved to the specified path to facilitate subsequent analysis and reference. Returning the result, the function returns the negative_positive_result list, providing the prediction labels for each image. These labels can be used for further automated processing and processing. By utilizing the detect_batch_deit function for image prediction and automation, the efficiency of determining serological test detection results is significantly improved. This function integrates the steps of initialization, image sorting, save path handling, and image processing loops, allowing for rapid and accurate image predictions and result generation.

In the present embodiment, the DeiT_meidu syphilis classification model detection results:

### (1) Trust Samples:

One-time detection results: 212 positive circles, with an accuracy of 95.75%; 200 negative circles, with an accuracy of 95.5%.

One-time detection results: 212 positive circles, with an accuracy of 95.75%; 200 negative circles, with an accuracy of 95.5%.

### (2) RPR Samples:

One-time detection results: 75 positive circles, with an accuracy of 90.66%; 77 negative circles, with an accuracy of 84.42%.

Two-time detection results: 35 positive circles, with an accuracy of 82.86%; 25 negative circles, with an accuracy of 80.0%.

Compared with the gold standard, the accuracy of negative and positive recognition for TRUST samples is between 93% and 96%, and the titer interpretation accuracy is between 90% and 92%. The accuracy of negative and positive recognition for RPR samples is between 86% and 89%, and the titer interpretation accuracy is between 85% and 86%.

The DeiT_meidu syphilis classification model has high computational efficiency. On average, detecting multiple samples within a single image takes only 0.6 seconds, whereas experienced inspectors and doctors take more than 3 seconds on average to interpret a single sample in the same image. Through the rapid computation of the algorithm, the speed and efficiency of detection can be improved. The DeiT_meidu syphilis classification model follows the process of detection-segmentation-preprocessing-classification-judgment, making the interpretation process of serological test detection more straightforward. Combined with the visualization interface of the mini-program, doctors can conveniently use this model for interpretation operations, thereby improving work efficiency. By establishing a unified discrimination standard through the DeiT_meidu syphilis classification model, the influence of subjectivity from inspectors on the test results can be effectively avoided. This model makes judgments through algorithmic methods, reducing the interference of individual experience and subjective judgment on serological test detection results, which is beneficial for doctors in clinical analysis and decision-making.

The application of the DeiT_meidu syphilis classification model can significantly improve the automation processing efficiency of serological test detection results. This model has the advantages of high computational capability, a simplified interpretation process, and the elimination of subjective influence.

The above provides an illustrative description of the present application and its embodiments, and this description is not restrictive. Without departing from the spirit or fundamental characteristics of the present application, the present application may be implemented in other specific forms. The figures shown represent only one embodiment of the present application, and the actual structure is not limited to this. Any figure reference numerals in the claims should not be construed as limiting the claims involved. Therefore, if an ordinary person skilled in the field, inspired by this, designs a structural form and implementation similar to those of the present technical solution without departing from the spirit of this creation and without inventive design, they should fall within the protection scope of this patent. Furthermore, the word “include” does not exclude other elements or steps, and the word “a” before an element does not exclude the presence of multiple such elements. Multiple elements stated in product claims may also be implemented by a single element through software or hardware. The terms “first,” “second,” etc., are used for naming purposes and do not indicate any specific order.

1. A titer processing method based on deep learning, used for automatic processing of non-treponemal serological test results, comprising:
obtaining a first image of the non-syphilis treponemal serological test results;
judging the clarity of the test image and outputting a second image;
applying a convolutional neural network on the second image for detecting circular targets and outputting the center coordinates and radius of the circular targets;
segmenting the second image according to the center coordinates and radius of the detected circular targets to extract circular target regions;
sorting the extracted circular target regions according to preset sorting rules;
using a convolutional neural network to classify the sorted circular target regions and outputting a negative or positive result;
calculating a corresponding titer of the negative or positive result;
the step of applying the convolutional neural network on the second image for detecting circular targets and outputting the center coordinates and radius of the circular targets comprises:
obtaining a training set containing non-treponemal serological test images, labeling the circular targets in the training set, and obtaining a labeled data set;
using the convolutional neural network as the detection model, freezing the feature extraction layer of the detection model, and modifying the output of the detection model as the center coordinates and radius;
using the labeled data set to train the detection model;
using the trained detection model to predict the input second image, and outputting the center coordinates and radius of the circular targets.

2. The titer processing method based on deep learning according to claim 1, characterized in that:
the step of judging the clarity of the first image and outputting a second image comprises:
converting the first image of the input non-treponemal serological test result into a grayscale image;
performing edge detection on the grayscale image and calculating the variance and maximum value of the edge image;
based on a preset variance threshold and maximum value threshold, judging whether the variance and maximum value meet the requirements of the second image;
if the variance is less than the preset variance threshold and the maximum value is less than the preset maximum value threshold, then confirming the first image as the second image and outputting the second image.

3. The titer processing method based on deep learning according to claim 1, characterized in that:
the step of segmenting the second image according to the center coordinates and radius of the detected circular targets to extract circular target regions comprises:
drawing a corresponding circular region according to the center coordinates and radius of the circular target;
using binarization processing, setting the circular region as foreground pixel values M1, and setting other regions as background pixel values M2, obtaining a binarized image;
performing morphological processing on the binarized image to remove noise regions to obtain a refined binarized image;
generating a corresponding mask according to the refined binarized image;
subjecting the mask to a bitwise AND operation with the original second image to extract the circular target region.

4. The titer processing method based on deep learning according to claim 1, characterized in that:
the step of sorting the extracted circular target regions according to preset sorting rules to obtain sorted circular target regions comprises:
calculating the center coordinates of each circular target region detected and segmented;
judging the rows where the circular targets are located according to the longitudinal difference between the center coordinates of adjacent circular target regions;
comparing the lateral difference between the center coordinates of circular target regions in the same row to determine the columns of the circular targets in each row;
generating a sorting index of the circular target regions according to row order and according to column order in the same row;
rearranging the order of the circular target regions according to the sorting index.

5. The titer processing method based on deep learning according to claim 1, characterized in that:
the step of using a convolutional neural network for classifying the sorted circular target regions and outputting the negative or positive result comprises:
obtaining a training set containing the sorted circular target regions and labeling the negative and positive labels;
using a pre-trained lightweight convolutional neural network as a classification model;
freezing the feature extraction layer parameters of the classification model, and modifying the fully connected layer output of the classification model to be a binary classification, representing the negative and positive results;
using the labeled data set to fine-tune the classification model and optimize the model parameters;
evaluating the fine-tuned classification model and calculating a prediction probability pi;
applying the trained classification model to predict the sorted circular target regions and outputting the corresponding negative and positive results.

6. The titer processing method based on deep learning according to claim 1, characterized in that:
the step of calculating the titer corresponding to the negative and positive results comprises:
inputting the negative or positive result of each circular target region;
setting counters of the number of negative circular targets N1, the number of weakly positive circular targets N2, and the number of strongly positive circular targets N3 of each sample;
iterating the negative and positive results of each input circular target region, and updating the corresponding counters according to the negative and positive results;
calculating the total number of circular targets N for each sample, the total number of circular targets being the sum of each counter;
judging the counter values, determining whether the sample is negative, weakly positive, or strongly positive according to interpretation standards of the non-treponemal serological test, and calculating the corresponding titer;
outputting the judged negative and positive results and the calculated titer.

7. The titer processing method based on deep learning according to claim 6, characterized in that:
when the number of negative circular targets N1 is equal to the total number of circular targets N, then the non-treponemal serological test result is judged to be negative, and the titer is Y1;
when the number of negative circular targets N1 is not equal to zero and the number of strongly positive circular targets N3 is equal to zero, then the non-treponemal serological test result is judged to be weakly positive, and the titer is the number of weakly positive circular targets N2 divided by the total number of circular targets N;
when the number of strongly positive circular targets N3 is not equal to zero, then the non-treponemal serological test result is judged to be strongly positive, and the titer is Y2.

8. A titer processing system based on deep learning, used for automatic processing of non-treponemal serological test results, comprising:
an image clarity judgment module, used for judging the clarity of an input non-treponemal serological test image;
a circular target detection module, using a pre-trained convolutional neural network for detecting circular targets in a clear image;
a circular target segmentation module, used for segmenting the clear image according to the detection results and extracting circular target regions;
a circular target sorting module, used for sorting the extracted circular target regions;
a circular target classification module, using a pre-trained convolutional neural network for classifying the sorted circular target regions and outputting negative and positive results;
a titer calculation module, used for calculating a corresponding titer according to the negative and positive results;
wherein, using the pre-trained convolutional neural network for detecting circular targets in the clear image comprises:
obtaining a training set containing non-treponemal serological test images, labeling the circular targets in the training set, and obtaining a labeled data set;
using the convolutional neural network as the detection model, freezing the feature extraction layer of the detection model, and modifying the output of the detection model as the center coordinates and radius;
using the labeled data set to train the detection model;
using the trained detection model to predict the input second image, and outputting the center coordinates and radius of the circular targets.
